(19) United States
(12) Patent Application Publication
(10) Pub. No.: US 2025/0104823 A1
(43) Pub. Date: Mar. 27, 2025

(54) MEDICAL DATA PROCESSING APPARATUS AND METHOD FOR PROCESSING MEDICAL DATA

(71) Applicants: Siemens Healthineers AG, Forchheim (DE); FRAUNHOFER-GESELLSCHAFT ZUR FÖRDERUNG DER ANGEWANDTEN FORSCHUNG E.V., München (DE)

(72) Inventors: Sven KOHLE, Erlangen (DE); Mathias NEUGEBAUER, Bremen (DE); Diego BARRIOS ROMERO, Osterholz-Scharmbeck (DE); Felix RITTER, Bremen (DE); Christoph BRACHMANN, Achim (DE); Sandra SIEGEL, Bremen (DE); Anita STEIN, Hannover (DE); Alexander ROESLER, Hannover (DE); Steffen WEICHERT, Wohltorf (DE); Felix NENSA, Essen (DE); Christian TIETJEN, Fuerth (DE); Gerhard KOHL, Neunkirchen am Brand (DE); Michael RUSITSKA, Forchheim (DE)

(73) Assignees: Siemens Healthineers AG, Forchheim (DE); FRAUNHOFER-GESELLSCHAFT ZUR FÖRDERUNG DER ANGEWANDTEN FORSCHUNG E.V., München (DE)

(21) Appl. No.: 18/888,688

(22) Filed: Sep. 18, 2024

(30) Foreign Application Priority Data
Sep. 21, 2023 (DE) 10 2023 209 247.6

(51) Int. Cl. G16H 15/00; G06F 40/30; G06T 7/00

(52) U.S. Cl. CPC G16H 15/00; G06F 40/30; G06T 7/0012

(57) ABSTRACT

Embodiments relate to processing of medical data, in particular image data of medical images and related medical reports for automated generation of a draft report for a newly recorded medical image. The image elements relating to corresponding documentation elements for a former medical image are mapped to image elements in the newly recorded medical image, and based on this mapping, an automated generation of the draft of the medical report of the newly recorded medical image is performed.

### CROSS-REFERENCE TO RELATED APPLICATION(S)

The present application claims priority under 35 U.S.C. § 119 to German Patent Application No. 10 2023 209 247.6, filed Sep. 21, 2023, the entire contents of which is incorporated herein by reference.

### FIELD

One or more embodiments of the present invention relate to a medical data processing apparatus, a method for processing medical data and a non-transitory computer program product for performing processing of medical data.

### BACKGROUND

Independent of the grammatical term usage, individuals with male, female or other gender identities are included within the term.

In the context of medical examinations, imaging methods have become very important. Such imaging methods may comprise, for example, ultrasonic images, radiological images, especially images from a computer tomograph or a magnetic resonance tomograph.

After capturing one or more images of an examination area of a patient to be examined, the images are analyzed and documented by a medical specialist, e.g. a radiologist. For this purpose, the specialist creates a medical report for an acquired image, in which he or she documents his/her observations in the related image. When a further image of the same patient, in particular of a same area of this patient is taken at a later time, the specialist creates a completely or partially new report, in which he documents his observations for the further image.

Such a generation of medical reports for each medical image requires a relative huge amount of time. Further, the specialist has to ensure that all relevant observations of an image are comprised in the related medical report. In many cases the specialist may also wish to refer to other images, for example images of a same examination area of the patient taken at earlier times. All this makes the generation of a medical report a very complex and time-consuming matter and requires a high degree of responsibility and is error prone. It needs to be assured that information is not lost.

### SUMMARY

Even though embodiments of the present invention are described mainly in connection with a documentation of computer tomography images, the present invention is not limited to such images. Moreover, embodiments of the present invention are also applicable to almost any other kind of medical images.

The inventors have identified a need for a way to process medical data in the context of medical images which can provide an at least partially automated generation of medical reports for medical images. In particular, the inventors ove identified a need for an apparatus and a method for processing medical data which can provide an automated assistance for generating medical reports up to a generation of a complete draft for a new medical report of a medical image.

Embodiments of the present invention provide a medical data processing apparatus, a method for processing medical data and non-transitory computer program product with the features of the independent claims. Further advantageous embodiments are subject matter of the dependent claims.

According to a first aspect, a medical data processing apparatus is provided. The medical data processing apparatus comprises a first storage device, a second storage device, an input device, a mapping device and a processing device. The first storage device is adapted to store image data for a plurality of medical images. Each medical image may comprise an associated time stamp. The second storage device is adapted to store at least one medical report, in particular a medical report for a corresponding medical image stored in the first storage device. Each medical report may comprise at least one documentation element for an image element of the corresponding medical image. The input device is adapted to receive a selection for a first medical image and/or a first medical report for the first medical image. The input device is further adapted to receive a selection for a second medical image. The mapping device is adapted to calculate a mapping between the first medical image and the second medical image. The processing device is adapted to determine at least one image element in the second medical image for which a documentation element is comprised in the first medical report. The determination is performed by using the mapping be-tween the first medical image and the second medical image. The processing device comprises a documentation module. The documentation module of the processing device is adapted to generate a documentation element, in particular a documentation element for a draft second medical report for the second medical image. A respective documentation element for the second medical report is automatically generated using the corresponding documentation element for the corresponding image element in the first medical report. To be more precise, two different instances of a documentation element may exist: a first documentation element in the first medical report and a second documentation element in the second medical report.

According to a second aspect, the method of processing medical data is provided. The method comprises storing image data for a plurality of medical images. Each medical image may comprise an associated time stamp. The method further comprises storing at least one medical report for a corresponding stored medical image. The medical report comprises at least one documentation element for each image element in the corresponding medical image. Further, the method comprises receiving a selection for a first medical image and/or a first medical report for the first medical image, and receiving a selection for a second medical image. The method further comprises calculating a mapping between the first medical image and the second medical image. Further, the method comprises determining an image element in the second medical image for which a documentation element exists in the first medical report. A position, size and/or shape of the image element in the second image may be determined using the mapping between the first medical image and the second medical image. The method further comprises generating a draft second medical report for the second medical image. A documentation element for the second medical report is automatically generated using the corresponding documentation element for the corresponding image element in the first medical report.

According to a further aspect, a non-transitory computer program product is provided. The computer program product comprises a computer program being loadable into a memory unit of a control device according to the first aspect and containing program code sections for causing the control device to execute the method of processing medical image date according to the second aspect when the computer program is executed in the control device.

A medical image in the context of this application may be any kind of image which has been acquired for medical purposes. For example, the medical image may relate to a particular part or area of a human body or to an (e.g. artificial) structure, like e.g. an implant or stent etc. For instance, an image may be a scan of a specific organ or an area with a lesion or disease. A medical image may be a two-dimensional (or three-dimensional) representation of a specific part of a body or anatomical part. The representation may be provided, for example, in a greyscale or a colored image. This purpose, the representation of the image, i.e. the individual pixels may be determined from corresponding image data.

Even though embodiments of the present invention are described in connection with medical images and medical diagnosis of a human being, the general concept of the present invention is also applicable to similar applications, for example in the context of medical diagnosis for animals or the like.

Image data related to a medical image may comprise data for specifying the individual pixels of the image as well as further data related to the image. The data for specifying the pixels may be provided directly, for example by providing greyscale or color values for the individual pixels. For this purpose, any appropriate standardized or proprietary image format may be used. Alternatively, it may be also possible to provide any kind of raw or intermediate data from which the pixel values for the medical image can be derived.

The medical image may be represented in a dataset. The dataset may comprise image data and/or further data. The medical image may e.g. be formatted in the DICOM format and may comprise a header section.

The image data may relate to a two-dimensional or three-dimensional representation of an imaged structure (anatomical or medical structure). In case that the image data relates to three-dimensional representation, such as a scan of a computer tomograph it may be possible to compute a two-dimensional image e.g. a cross-section through the three-dimensional model.

The further data of the image data may specify, for example, any kind of additional information, related to the medical image, e.g. metadata. The further data may be part of the header section. For instance, the further data may relate to technical aspects in connection with the medical image or the process when acquiring the medical image. The further data of the image data may specify, e.g. a timestamp, in particular an indication of time when the related medical image has been captured. The further data may also specify the device which is used for capturing the medical image and/or further information, e.g. a configuration or setting of the respective device when capturing the medical image. The further data may also comprise information specifying the person/patient from which the image has been captured, a desired purpose for capturing the image, etc. However, it is understood, that the further data of the image data may also comprise any other kind of appropriate information, in particular technical and/or administrative information related with the medical image.

Beyond the image data with the data of the medical image and/or the further data (auxiliary) data, a medical report may be generated and provided in association with the related medical image. In contrast to the further or auxiliary data related to the image data, the related medical report provides medical details in connection with the related medical image.

The medical report is typically related to at least one medical image. Thus a first report relates to a first medical image and a second report relates to a second medical image. The first and second medical image may be acquired from the same or from different modalities. The first and second medical image may be acquired at different time stamps. It is, however, also possible, that the first and the second medical image are acquired in a corresponding time phase with different settings (e.g. different resolution, different perspective, different field of view etc.).

The medical report may comprise one or more documentation elements. A documentation element may thus be a part or portion in the medical report. A documentation element may be provided in textual and/or verbal form. A documentation element typically relates to the corresponding medical image or a part or feature therein, in particular relates to an image element in the medical image. For example, a documentation element may be a textual passage, specifying the tumor, which is visible in the corresponding medica image, e.g. specifying its cells, size, structure, etc.

Each documentation element may relate to a particular feature of the related medical image. For example, a documentation element may characterize a particular property of a feature in the medical image. A documentation element may specify, for example, a size, volume, shape, position and/or intensity of any other characteristic of an element shown in the related medical image. Especially, in case when multiple corresponding medical images have been captured at different points in time, a documentation element for a particular feature in the medical report may also refer to variations of the respective feature with respect/reference to another medical image, e.g. development of a tumor over time in different medical images, acquired at different points in time. Alternatively or in addition, multiple corresponding medical images may refer to the same time frame and/or anatomical structure but may stem from different modalities.

A feature (e.g. a lesion, like a tumor or a calcification or a segmented organ) in a medical image may be represented by an image element in the medical image. Such an image element may be, for example, an image area which has been selected manually by the user. Additionally or alternatively, it may be also possible that an image element for a particular feature may be selected automatically. For instance, an appropriate device may perform an analysis of the image data in order to identify one or more predetermined properties. For example, it may be possible to automatically identify an image area relating to a specific organ, a lesion, a tumor, etc. This may be done e.g. by using automatic image analysis and/or segmentation algorithms. The approach for identifying such an image area may be performed based on any appropriate schema. For example, an analysis employing artificial intelligence, neural networks or the like may be used. However, it may be also possible to perform such analysis by evaluating the values of the pixels in the medical image in order to identify a borderline, an area of the like. After an automated determination of an image area is performed, it may be a further possible that a user manually may adapt or adjust the result of the automated determination process.

If a new (also mentioned herein: second) medical report is to be created for a new/further (also mentioned as second) medical image, this new medical report preferably should comprise documentation elements corresponding to respective documentation elements in a former medical report relating to a former medical image. Thus, it is to be noted, that the term “documentation element” in this application has to be construed as comprising two different instances or occurrences: a first documentation element in the first medical report and a second documentation element in the second medical report. Further, it is to be noted, that “documentation element” is to be construed as “at least one documentation element”. With other words, where a documentation element (be it a first one or a second one does not matter) is mentioned, it may generally refer to one or more documentation elements.

Hence, the person creating such a new medical report has to consider the former medical image and the related medical report, identify documentation elements in the former medical reports, the related features in the former medical image, and determine the corresponding features in the new medical image. Then, the person and/or an electronic module has to create a respective new documentation element in the new medical report. For this purpose, the person and/or an electronic module has to analyze the respective feature in the new medical image and compare this feature with the related feature and/or the documentation element in the former medical report. Based on this analysis, the person and/or an electronic module can create the respective element in the new medical report. Usually, the first medical image (and corresponding first medical report) has a time stamp which is older than a time stamp of the second medical image, because the second medical image is typically acquired later. However, also other application scenarios may arise.

For example, it is also possible to generate a report for a former image, based on a new image with a new report. “New” in this sense means, an image having a time stamp which is younger than the time stamp of a former image (acquired earlier, and thus having an older time stamp).

The documentation element of a first medical report may be equal, similar or different to the corresponding documentation element of a second medical report. For example, the first documentation element in the first medical report may refer to the kind of lesion or structure, visible in the first image and the second documentation element in the second medical report may be equal to the first documentation element as is still refers to the same kind of lesion. However, if the first documentation element in the first medical report relates to the size of a lesion, then the second documentation element may be different from the first one, as the size may be different, if e.g. a tumor has become larger.

For conventional approaches, a user, e.g. a medical specialist, has to perform the above-described tasks manually. On the one hand side, performing all these tasks manually requires a lot of work. On the other hand side, the user has to ensure that he indeed considers all documentation elements of a former medical report when creating a new medical report for a new medical image.

In view of these objectives, it is an idea of embodiments of the present invention to provide technical devices, apparatuses and/or means for assisting the user when generating a new medical report and to provide an approach for an at least automated generation of a draft report for a new medical image.

For this purpose, image data for a plurality of medical images and medical reports relating to at least some of the medical images may be provided. The image data may be stored, for example, in a first storage device, and the medical reports may be stored, for example, in a second storage device. The first and second storage devices may be any kind of appropriate storage devices such as local storage devices, e.g. hard disk drives, solid state drives, network storage (NAS). Furthermore, it may be also possible to store the image data and/or the medical reports in storage devices of a cloud service or the like. Even though the terms first and second are used for the storage devices of the image data and the medical report, it may be also possible to store the image data and the medical reports in a same, common storage device. In this case, the image data and the medical report, may be stored, for example, in separate files, separate folders or the like. In each case, a clear, unique relationship between the image data and the related medical report may be provided. For example, the image data may comprise a reference to the related medical report and/or a medical report may comprise a reference to the related image data. However, it is understood, that any other appropriate scheme may be used, too.

In order to perform an assisted or automated generation of a new medical report, a user may specify a first image and/or a first medical report. Further, the user may specify a second medical image for which the assisted or automated generation of the medical report shall be performed. The selection of the respective images or reports may be performed, for example, via an appropriate input device. Such an input device may be, for example, any appropriate device which is in the position to receive the required input from the user. Especially, a graphical user interface or the like may be used. For example, the user may enter an identifier, e.g. a specific name, in order to select a medical report or a medical image. In particular, it may be possible that the input device provides multiple possibilities for available medical images and reports, and the user may select a desired image of report. This may be performed, for example via a touchscreen, a computer mouse of in any other appropriate manner. Furthermore, it is also possible to perform the selection of the images or reports by voice commands or the like.

After selecting the second medical image for which a medical report shall be created, and selecting the first medical image and first medical report, which shall serve as a basis of for creating the new medical report, a mapping between the first medical image and the second medical image is performed.

The mapping determines a relationship between the first medical image and the second medical image. For example, the mapping may specify a transformation between positions in the first medical image and the second medical image. In one possibility of such a mapping, a relationship between pixels in a coordinate system of the first medical image and the pixels in a coordinate system of the second medical image may be determined. The mapping may be applied to the medical images, so that a first medical image is mapped to a second one. Alternatively or in addition, the mapping may be applied to image elements in the respective images, so that a first image element is mapped to a second image element. Based on such mapping, it is possible to represent or show a development of the body structure to be examined over time, like e.g. stages of development of a tumor or a decrease of an inflammatory reaction.

Further, it may be possible to perform a mapping of image elements in the first medical image to corresponding image elements in the second medical image. In particular, such a mapping of image elements may be performed for image elements relating to documentation elements in the first medical report. In order to perform such a mapping, any appropriate existing or upcoming approach for a mapping of the first and second medical image may be applied. Such mapping may be performed, for example, by a mapping device. A mapping may be based on a segmentation algorithm.

In this way, it is possible to identify image elements in the second medical image in particular those, for which a documentation element in the first medical report exists. For example, based on the mapping, for each image element in the first medical image relating to a corresponding documentation element in the first medical report, a corresponding image element in the second medical image may be determined. This determination of the related image elements in the second medical image may be performed, for example, by an appropriate processing device.

After identifying image elements in the second medical image which correspond to the related image elements in the first medical image, a draft for a new medical report for the second medical image may be generated. For this purpose, for each documentation element in the first medical report, a corresponding documentation element in the new medical report may be created. For such a “new” or “second” documentation element in the “new” or “second” medical report (second here refers to the medical report to be generated), a reference to the corresponding image and in the second medical image may be generated in the new medical report. Further to this, it may be also possible to make a reference in the documentation element of the new medical report to the documentation element in the first medical report and/or the related image element in the first medical image. It may be also possible to automatically perform an analysis of the related image element in the second medical image in order to determine one or more predetermined properties of this image element. For example, it may be possible to determine a size, volume, length, shape and/or any other property automatically. Such automatically determined properties of the image element in the second medical image may be also added to the expected documentation element in the new medical report. If necessary, further operations for the automated generation of the draft new medical report may be also performed. All these operations may be performed, for example, by the processing device.

The mapping device and/or the processing device may be implemented, for example, by any kind of appropriate computing device. For example, the mapping device and/or the processing device may comprise a processor and a related memory storing program instructions in order to perform the desired operations. The first and second storage device, the input device, the mapping device and the processing device may be communicative the connected with each other in order to perform the required data exchange.

In a possible embodiment, the apparatus further comprises an output device. The output device may be adapted to output the first image, the corresponding first report for the first image, and/or the second image and the draft for the second report. The input device may be adapted to receive user input for a selection of an image element in the output images and/or a documentation element in the output reports. The processing device may comprise a processing module adapted to identify an image element corresponding to the selection in each of the first image and the second image, and to identify a documentation element corresponding to one of the first report and the second report. The output device may be, for example, a display such as an OLED or TFT screen. However, any other appropriate device for displaying the required information, in particular the representations of the medical images and the medical report, may be possible, too. In particular, the output device may be part of a system for providing a graphical user interface (GUI). For example, the output device may comprise a touch screen for displaying information and receiving user input. However, it may be also possible to receive user input via a keyboard, a computer mouse, voice commands, etc.

In a possible embodiment, the input device is adapted to receive an adjustment for a mapping of an image element in the first image and an image element in the second image. In such a configuration, the processing device may be adapted to adjust the mapping between the image elements in the first image and the second image based on the received adjustment. In order to adjust the mapping between the image elements in the first and second medical image, any appropriate approach may be used. For example, a GUI may receive a user command for selecting an image element and for adapting/adjusting the image element in one of the images. For this purpose, the user may adjust a position, size, shape and/or any other feature of a respective image element. In this way, the user may perform, for example, a fine-tuning of the automated mapping between the image elements in the first and second medical image.

In a possible embodiment, the apparatus further comprises a third storage device. The third storage device may be adapted to store the mapping between the first image and the second image and/or to store a mapping between a pixel in the first image and a corresponding pixel in the second image. The stored mapping, in particular the mapping relationship after further adjustments by a user, may be used for improving the mapping approach between medical images. For example, the information of the adjusted mapping may be used as training data for a neural network, in particular a neural network applying the mapping. However, it is understood, that any other approach may be possible, too.

In an embodiment, the first, second and/or third storage may be implemented in one common storage or platform or may be provided separately.

In a possible embodiment, the apparatus comprises an analysis module. The analysis module may be adapted to determine at least one property of the image element in the second image. In this configuration, the documentation module may be adapted to generate the draft second report using the determined at least one property of the image element. Such a property of an image elements may be, for example, a length, size, volume, shape, density and/or any other appropriate feature for characterizing the related image element. For example, the analysis may be performed, based on an appropriate image processing algorithm. The algorithm may identify, for example, a border of the feature in the respective image element and determine one or more characteristic feature related to the image element within this border, in particular via a segmentation algorithm. However, any other approach, in particular an approach using artificial intelligence, neural networks or the like may be possible, too.

In a possible embodiment, the analysis module may be adapted to determine the at least one property to be determined using a semantic analysis of the corresponding documentation element in the first report. For example, the semantic analysis may identify a keyword in the related documentation element and/or in the metadata of the image. Based on this identified keyword, the analysis may perform an automated determination of the feature relating to such a keyword. For instance, if the semantic analysis identifies the term “length” in the related documentation element of the first medical report, the analysis performed by the analysis module may automatically determine a length of the corresponding image element in the second medical image. The determined lengthy may be automatically inserted in the respective documentation element of the newly generated second medical report. However, it is understood, that this approach may be also performed for any other keyword and any other kind of semantic analysis.

In a possible embodiment, the apparatus comprises an evaluation device. The evaluation device may be adapted to identify a related image element in a plurality of medical images and a respective property of each identified image element. Further, the evaluation device may be adapted to determine a progression of the property of the image element as a function over time, i.e. over the related time stamps. In this way, a progression of the feature relating to an image element in multiple medical images over the desired period of time can be automatically determined and, if necessary, visualized. Moreover, it is also possible to use this progression or a description relating to such a progression in a related documentation element in the newly generated medical report. In particular, it is possible that the analysis of this progression referring to further medical images, in particular further medical images in the past, may be performed automatically without the need of manually opening the respective medical images by the user.

In a possible embodiment, the documentation module is adapted to generate a respective documentation element in the second report for each image element in the first image and/or for each documentation element in the first report. By automatically generating the respective documentation elements in the (newly generated) second medical report, it can be ensured that the new report comprises a documentation element for each image element/documentation element of the first report. In this way, the completeness of the new medical report can be ensured.

In a possible embodiment, the apparatus comprises a first data interface. The first data interface is adapted to be coupled to a medical diagnostic imaging system. The first data interface may be further configured to receive data of medical images. Further, the first data interface may be configured to store the received data in the first storage device. In this way, the apparatus for processing the medical data and in particular for automated generation of medical reports can be easily implemented in a medical system, in particular a system dealing with medical images.

In a possible embodiment, the medical images may comprise ultrasonic images or radiological images, in particular images from a computer tomograph, a positron emission tomography and/or from a magnetic resonance tomograph. However, it is understood, that any other kind of medical image, in particular medical images for diagnosis purposes may be possible, too.

In a possible embodiment, the apparatus comprises a second data interface. The second data interface may be adapted to provide data of a medical image and a corresponding medical report to an external processing device. Additionally or alternatively, the second data interface may be configured to receive information for an image element of a medical image or of a documentation element of a medical report. The second data interface may be further configured to automatically add the received information to the corresponding image and/or report. In this way, a further a data exchange with additional devices, in particular additional devices of a medical system dealing with images, can be implemented. By receiving further information and automatically adding this information into a documentation element of the medical report, the assisted automated generation of the medical report can be further improved.

In a possible embodiment, the apparatus further comprises a collation device. The collation device is configured to identify at least one documentation element in a first medical report. The identification of the at least one documentation element may be performed using an analysis of the first medical report. In particular a semantic analysis may be performed. The collation device may be further configured to determine a corresponding image element in the first medical image for each of the identified documentation elements. In this way, it is possible to use medical images and corresponding medical report even if no links or relationships between the first medical image and the first medical report are available in the original information. Moreover, the collation device can perform an automated process for a referencing between the first medical image and the first medical report. Thus, it is possible to identify appropriate image elements in the first medical image to which the first report refers to. In this way, it is possible to use further data sources, in particular further medical images and medical reports which are not originally generated in the apparatus for processing the medical data and thus, there is no relationship between medical images and reports in the original data sources.

In a possible embodiment, the image elements are positioned within the first or second medical image at a related (embedded) location. Thus, the image elements may be limited to a predetermined area within the medical image. Additionally or alternatively the image elements may comprise an indication of a location, an organ, a lesion and/or a size indicator.

In a possible embodiment, the mapping between the first medical image and the second medical image comprises mapping two-dimensional or three-dimensional coordinates. In this way, the mapping operation may provide a transformation scheme based on which pixels, image areas or the like in the first medical image can be easily assigned to corresponding pixels, image areas or the like in the second medical image. Hence, a fast and easy scheme for specifying the relationship between medical images or image elements can be realized. It can be possible to save a determined mapping between medical images, and to re-use this mapping at a later point in time. Thus, computational resources can be saved.

### BRIEF DESCRIPTION OF THE DRAWINGS

In the following detailed description of the figures, non-limiting exemplary embodiments with the features and further advantages thereof will be discussed with reference to the drawings. In the figures:

FIG. 1 shows a schematic diagram of a graphical interface provided by an apparatus for processing medical data according to an embodiment;

FIG. 2 shows a schematic block diagram of an apparatus for processing medical data according to an embodiment; and

FIG. 3 shows a flow diagram illustrating a method for processing medical images according to an embodiment.

### DETAILED DESCRIPTION

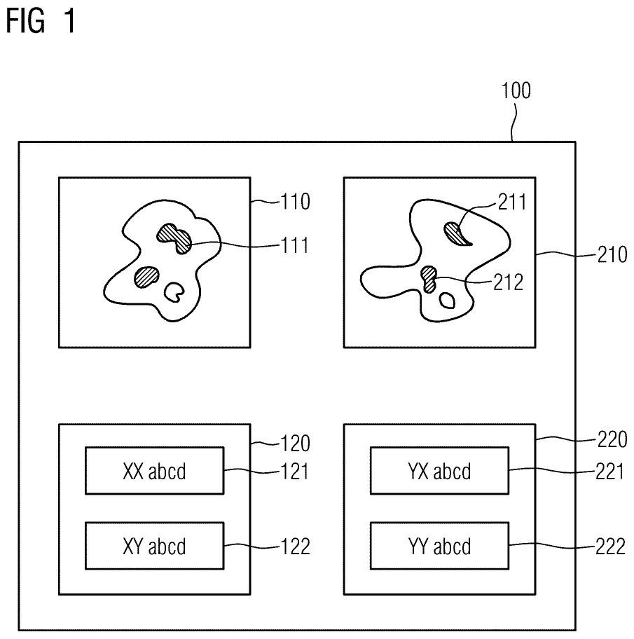

FIG. 1 shows a schematic diagram of a graphical interface 100 which illustrates some main elements in connection with a processing of medical data for an automated or assisted generation of a medical report.

For example, a new (second) medical report 220 may be generated for a related and/or corresponding (second) medical image 210. For this purpose, the generation of a draft for the new (second) report 220 may be performed automatically based on another (first and/or e.g. earlier) medical report 120 and a first medical image 110 related to this first medical report 120. In the following, the other medical image is referred to as first medical image 110, and the related further or other medical report is referred to as first medical report 120. Accordingly, the new medical report, which shall be generated is referred to as second medical report 220, and the related image for this second medical report is referred to as second medical image 210.

As can be seen in FIG. 1, the first medical report 120 may comprise one or more documentation elements 121, 122. Each documentation element may relate to a corresponding image element 111, 112 in the first medical image 110. An image element 111, 112 may be, for example, an indication of a specific position, area and/or volume in the medical image 110. For example, the image element 111, 112 may be specified by a user when creating the first medical report 120. An image element 111, 112 may specify any kind of feature or characteristic in the related medical image 110. For example, an image element may relate to an organ, a lesion, a tumor or any other property in the medical image 110. The image element 111, 112 may be indicated by a point, a line, an arrow, a circle, a polygon or any other appropriate graphical or non-graphical element.

The first medical report 120 which refers to the first medical image 110 may comprise one or more documentation elements 121, 122. For example, each documentation element 121, 122 may refer to a corresponding image element 111, 112 in the related medical image 110. For example, a documentation element may specify “The image element xx shows a tumor with the size of 10 mm.” Accordingly, such a documentation element 121, 122 may comprise at least a reference to an image element 111, 112 and a description referring to this image element 111, 112.

On the right-hand side of FIG. 1, a second medical image 210 and a second medical report 220 are shown. For example, the second medical image 210 may be a newly acquired medical image for which a new (or second) medical report 220 shall be generated. For this purpose, and automated or at least assisted approach for drafting such a new, second medical report 220 can be applied.

In order to perform such a (semi-) automated generation of a draft for a second medical report 220, a mapping between the first medical image 100 and the second medical image 210 is performed. The mapping may comprise, for example, a transformation scheme between pixels or positions in the first medical image 110 and the corresponding pixels or positions in the second medical image 210. Based on this mapping, it is possible to automatically identify image elements 211, 212 in the second medical image 210 which correspond to related image elements 111, 112 in the first medical image.

Further to the identification of image elements 211, 212 in the second medical image 210 based on the mapping scheme, it may be possible to perform additional operations in order to identify characteristic regions or areas in the second medical image 210 with features or characteristics corresponding to the features or characteristics of the related image element 111, 112 in the first medical image 110. In this way, the identification of appropriate image elements 211, 212 in the second medical image 210 can be further improved.

After having identified the image elements 211, 212 in the second medical image 210, an automated generation of a draft for the second medical report 220 can be performed. For this purpose, related documentation elements 121, 122 in the first medical report 120 can be identified, and corresponding documentation elements 221, 222 in the draft for the second medical report 220 can be generated.

In this connection, the documentation elements 221, 222 in the second medical report 220 may be adapted based on the properties of the related image elements 211, 212 in the second medical image. For example, if a documentation element 121, 122 in the first medical report 120 describes a size of an image element 111, 112 in the first medical image 110, the size of the related image element 211, 212 in the second medical image can be automatically determined and the result of this determination can be inserted in the related documentation element 221, 222 in the second medical report 220. In this way, an automated generation of the new, the second, medical report 220 for the second medical image 210 can be realized.

A user may can read the draft for the second medical report 220, evaluate it and/or adjust the automatically generated second medical report 220, if necessary. For example, the user can directly edit the draft of the second medical report 220. This may be performed, by any appropriate devices, apparatuses and/or means, for example using a keyboard, a computer mouse or voice recognition.

Further, the user may also edit or adjust the automatic selection of the image elements 211, 212 in the second medical image 210. After adjusting an image element 211, 212 in the second medical image 210, the related properties of the image element 211, 212 may be automatically determined, and the respective documentation element 221, 222 in the second medical report 220 may be also adapted accordingly. For example, if a user adjusts the shape or extension of an image element 211, 212 in the second medical image 211, a new size of this image element 211, 212 may be determined and the newly determined size may be automatically inserted in the related documentation element 221, 222.

A user may also add further documentation elements 221, 222 in the second medical report 220. For example, the user may identify an existing or new image element 211, 212 in the second medical image 210. In response to this, a new documentation element 221, 222 may be generated in the second medical report 220. Further to this, the related position or area in the first medical image 110 may be also determined and shown. This determination of an image element in the first medical image which corresponds to the newly selected image element 211, 212 in the second medical image 210 may be also performed, for example, based on the determined mapping between the first medical image 110 and the second medical image 210.

In the following, a schematic concept of an apparatus 1 for processing medical data, especially for processing medical data in order to generate medical reports, will be described.

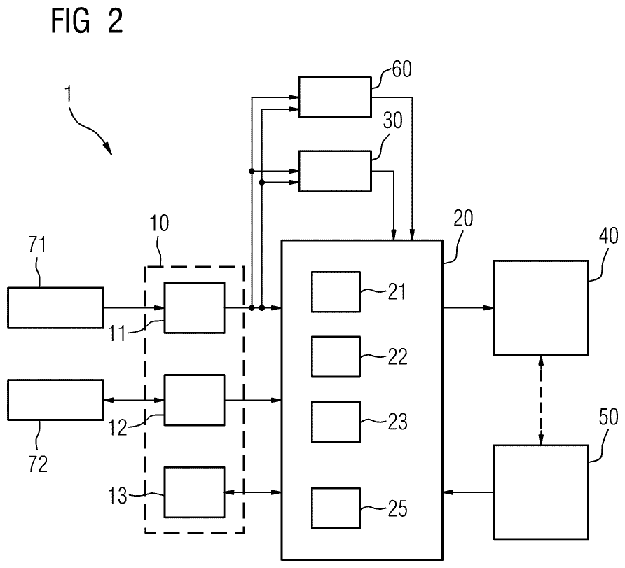

FIG. 2 shows a schematic block diagram of an apparatus 1 for processing medical data according to an embodiment. The apparatus 1 may comprise a first storage device 11 and a second storage device 12. Optionally, the apparatus 1 may comprise further storage devices, for example a third storage device 13. Even though the storage devices are shown as separate storage devices 11, 12, 13, it may be also possible that the individual storage devices 11, 12, 13 are realized at least in part by individual storage areas of a common storage device 10.

The first storage device 11 may store image data, in particular image data of multiple medical images. A medical image in this context may be any kind of image which provides a representation for medical/diagnostic purposes. For example, the medical image may be an ultrasonic image or a radiological image. In particular, the radiological image may be an image obtained from a computer tomograph and/or a magnetic resonance tomograph. However, any other appropriate image for medical purposes, like a positron emission tomography or ultrasonic device, may be possible, too.

The image data for the medical images may be obtained, for example, from a medical diagnostic imaging system. Such a medical diagnostic imaging system may comprise, for example the above-mentioned computer tomograph, positron emission tomography, magnetic resonance tomograph or ultrasonic device. Any other appropriate medical imaging system may be possible, too. For a data exchange between the medical imaging system and the apparatus 1 for processing the medical data, the apparatus 1 may be connected to the medical imaging system by a first data interface 71. In particular, the first data interface 71 may receive image data from the medical imaging system and store the received image data in the first storage device 11. For this purpose, any appropriate communication scheme such as ethernet or the like may be possible.

The second storage device 12 may store data of medical reports, in particular medical reports for corresponding medical images are stored in the first storage device 11. The medical reports may be, for example, medical reports as already described above in connection with FIG. 1. Accordingly, each medical report comprises one or more documentation elements relating to corresponding image elements of a related medical image stored in the first storage device 11.

A second data interface 72 may be further provided for a data exchange between the apparatus 1 for processing the medical data and any kind of further external processing device. Such a further external device may be, for example, a remote storage device, a cloud service or the like. Furthermore, a further external device may be a further device of a medical infrastructure, e.g. a device providing further medical information such as blood values, a pathological diagnosis, etc.

Furthermore, the further device of the medical infrastructure may be a device providing medical information, for example, a medical report or a device requesting medical information, for example a medical report stored in the second storage device 12 and/or a medical image stored in the first storage device 11. The communication between the further external processing device is the apparatus 1 for processing the medical data via the second data interface 72 may be also performed based on any appropriate communication scheme, for example, a network communication using ethernet.

The apparatus 1 for processing medical data may further comprise an output device 40. For example, the output device 40 may be a computer screen such as an OLED or TFT display. However, any other appropriate device for outputting graphical content may be possible, too.

Further, the apparatus 1 for processing medical data comprises an input device 50. The input device 50 may be any kind of input device for receiving user input. For example, the input device 50 may comprise a keyboard, a computer mouse or a microphone for receiving voice commands. Input devices 50 and output device 40 may be realized, for example, via a touchscreen or the like. In particular, a graphical user interface using the output device 40 and the input device 50 may be used for interacting with a user.

The apparatus 1 for processing medical data further comprises a processing device 20. The processing device 20 may read image data stored in the first storage device 11, process the image data and cause output device 40 to display a medical image corresponding to read image data. Further, the processing device 20 may read data of a medical report stored in the second storage device 12 and cause the output device 40 to display the content of the respective medical report. In particular, a medical image and the corresponding medical report may be provided in any appropriate relationship by the output device 40. For example, the related medical report may be displayed below a displayed medical image. However, any other appropriate scheme for providing a medical image and a related medical report may be possible, too.

In order to generate a new medical report or to modify or adapt a medical report for another (i.e. new or second) medical image, a user may select the medical image. In the following, this medical image is denoted as second medical image. For this purpose, the user may use, for example the graphical user interface provided by the output device 40 and the input device 50.

Further, the user may select a further medical image, in particular a medical image recorded at an earlier time for a corresponding or even the same purpose. In the following, this further medical image is denoted as the first medical image Thus, the user can refer to this first image, when creating the new (second) medical report. Additionally or alternatively, the user may select a medical report created at an earlier time, in particular a medical report relating to an image acquired at an earlier time for the same purpose. The following, this medical report is denoted as the first medical report. If the user selects the first image relating to an earlier time, processing device 20 may automatically identify a related first medical report for this first medical image. Alternatively, if a user selects a first medical report created at an earlier time, the processing device 20 may automatically determine the related first medical image for this first medical report. After this, the processing device 20 may output the first medical image and the related first medical report together with the second medical image for which a second medical report shall be created.

As already described above, a medical report may comprise one or more documentation elements, in particular documentation elements referring to the related image elements in the corresponding medical image. Therefore, processing device 20 may determine the individual documentation elements in the first medical report and the corresponding image elements in the related first medical image. The image elements and/or documentation elements may be indicated, e.g. highlighted, if required.

The processing device 20 may comprise a processing module 22 for identifying an image element corresponding to a user selection. Processing module 22 may identify an image element corresponding to a user input/selection of the user. For example, the user may click on a particular image element or a documentation element related to an image element. If the user selects a specific documentation element, the corresponding image element in the related medical image may be highlighted. Alternatively, if the user selects a specific image element in a medical image, the corresponding documentation element in the medical report may be highlighted.

In the following and automated or assisted generation of a new medical report based on the selected former medical report and the corresponding former medical image will be described.

After selecting the second medical image for which the new, second medical report shall be created and further selecting/identifying a first medical image and a first medical report, a mapping between the first medical image and the second medical image is performed. For this purpose, a mapping device 30 may be provided in the apparatus 1 for processing medical data. The mapping may use any appropriate existing or upcoming method for determining a relationship between the first medical image and the second medical image. For example, the mapping may determine a transformation scheme, specifying a relationship between a position of pixel in the first medical image and a position of a corresponding pixel in the second medical image. In particular, the mapping may specify a relationship between positions in the first medical image and positions in the medical image such that corresponding pixel related to same features, e.g. an organ, a lesion, a tumor, a bone, a cavity, a cyst, etc. The mapping may be performed, for example using artificial intelligence, neural networks, etc. However, it is understood, that any other appropriate approach may be possible, too.

After determining the mapping between the first medical image and the second medical image, processing device 20 may identify one or more image elements in the second medical image, which correspond to respective image elements in the first medical image. If wanted by the user, the identified image elements in the second medical image may be indicated, e.g. highlighted, in the second medical image displayed on output device 40.

Additionally or alternatively, the processing device 20 may identify the individual documentation elements of the first medical report relating to the first medical image. Based on the determined documentation elements of the first medical image, processing device 20 may determine the related image elements in the first medical image. Further, the processing device 20 may determine the corresponding image elements in the second medical image based on the determined mapping between the first medical image and the second medical image.

In order to generate a draft for the new, i.e. the second, medical report, the processing device 20 may comprise a documentation module 21 which may automatically generate a new documentation element in the second medical report for each documentation element in the first medical report and/or for each identified image element in the second medical image.

In particular, the documentation module 21 may generate (second) documentation elements for the second medical report which refer to the related image elements in the second medical image. If appropriate, a (second) documentation element in the second medical report may be also comprise a reference to the related (first) documentation element in the first medical report and/or to a related (first) image element in the first medical image.

Further to this, the documentation element in the draft for the second medical report may be automatically adapted according to features/properties of a related image element in the second medical image. For this purpose, processing device 20 may comprise an analysis module 23. Analysis module 23 may perform an analysis of an image element relating to a documentation element in order to determine a property of the image element which shall be documented or specified by the documentation applicant. The desired properties may be determined, for example, based on a semantic analysis of the respective documentation element. For example, if a documentation element comprises an expression like “the size is”, analysis module 23 may automatically determine that the desired feature is a size and thus, an analysis of the related image element is performed in order to determine the size of the image element is performed. Is understood, that the feature “size” is only an example, and is also possible to determine any other kind of feature of property such as volume, density, shape, etc.

In the foregoing description, an image element in the second medical image is automatically determined based on a mapping between the first and second medical image. Even though such an automated mapping may already provide very good results, it may be desirable to adapt and automated detected image element. For this purpose, a user may manually adjust the automatically detected image element. For example, the user may use the input device 50 for adjusting the image element. User may use, for example, a computer mouse, a touch screen or any other appropriate component of input device 50 for adapting an image element in a displayed medical image, in particular the second medical image. For example, the user may move a position of the image element, adapt a border of the image element, add further features such as an arrow or line, change the shape of the image element or perform any other appropriate amendment.

After adapting an image element by the user, the processing device 20, in particular analysis module 23 may perform a further analysis in order to determine the desired feature based on the adapted image element. For example, if the user changes the shape or size of an image element, analysis module 23 may automatically determine the new size of the amended image element.

After this, the newly determined feature may be automatically adapted in the related documentation element of the medical report.

In case that the user may adjust or change an automatically determined image element in the second medical image, the adjusted relationship between an image element in the first medical image and the second medical image may be also stored. For example, the mapping, in particular the adjusted mapping between the first medical image and the second medical image may be stored in a further storage device, for example a third storage device 13. In this way, the mapping is available at a later point in time. Hence, there is no need for a new mapping process, if the second medical report shall be amended later. Further to this, the mapping, in particular the adjusted mapping stored in the third storage 30 may be also used, for example, for training a neural network. In this way, the neural network for performing the mapping operations can be further improved based on the additional data provided by the third storage device 13.

After the draft for the second medical report has been automatically prepared by the processing device 20, in particular, by the documentation module 21, the user may further amend this draft report manually, if necessary. For example, the user may add further text such as explanations, diagnostics etc. to one or more documentation elements. For this purpose, the user may enter the desired amendments or supplements via the input device 50. In particular, the user may enter text by a keyboard or the user may use voice commands in order to modify the draft for the second medical report. In this connection, it is possible that a user may select a specific documentation element or image element, and processing device 20 may automatically cause highlighting all relevant documentation elements in the first and second medical report as well as the related image element in the first and second medical image. Accordingly, the user can easily recognize all relevant information provided by the output device 40.

In the foregoing, it has been assumed so far that there exists a medical image with one or more image elements and the corresponding medical report with one or more documentation elements refers to these image elements. However, in some cases it may be desirable to import medical images and medical reports directly, for example, from external sources. In this case, there may not exist any relationship between a medical image and a corresponding medical report. For the above-described automated generation of medical reports, however, it is desirable to establish such a relationship between medical images and reports. For this purpose, processing device 20 may comprise a collation device 25. The collation device 25 may perform an analysis of a medical report and related medical image in order to identify one or more documentation elements in a medical report. This analysis may be performed, for example, based on a semantic analysis of the respective medical report. Further, the collation device 25 may a perform an analysis of the related medical image in order to determine for each documentation element a corresponding image element. This relationship between an identified documentation element and the corresponding image element may be add to the respective medical report and/or medical image.

The described concept of automatic determination for features of corresponding image elements in multiple medical images may be also expanded into a comparison of more than two related medical images. For example, it may be possible to determine a corresponding image element in multiple medical images recorded at different points in time. In this case, it may be further possible to automatically determine one or more properties of such corresponding image elements for the multiple medical images and to provide information about a development of such a feature. For instance, it may be possible to determine a size, volume, etc. of a specific element in a series of images. In such a case, it may be also possible to enrich a documentation element in the second medical report by adding information with regard to this development. For example, it may be possible to at information about the individual values for multiple former medical images. Further, it may be also possible to determine a rate of control or reduction and to include this determined rate to a respective documentation element in the second medical report.

Such an analysis of a development with regard to a particular image element may be also performed in a separate task, for example before manually adapting a respective documentation element to this image element by a user. For example, apparatus 1 for processing medical images may further comprise an evaluation device 60 for performing such an analysis of the features relating to an image element in a serious of multiple medical images. For this purpose, a user may select a specific image element or alternatively a documentation element related to image element. Further, the user may select a set of multiple medical images. Alternatively, the period of multiple medical images may be selected automatically. For example, a user may specify a period of time, and evaluation device 60 may automatically select appropriate medical images for this period of time. However, any other appropriate approach for selecting multiple images for such an analysis may be possible, too.

After this, evaluation device 60 may identify at a corresponding image element in each of the selected medical images. For this purpose, the above-described mapping between the medical images may be applied in order to identify the desired image areas. Further, an automated analysis of the identified image area in the multiple medical images is performed in order to determine one or more values for one or more specific features of the image elements in all the medical images. After determining the desired values for the image elements in all the medical images, a progression over time of the respective values may be determined. The determined progression may be provided to the user, for example by displaying the results on output device 40. Further, it may be also possible to automatically add the determined progression or one or more information which are derived from this progression to a documentation element in the second medical report.

Even though mapping device 30 and evaluation device 60 are described as devices separate from processing device 20, it may be also possible to include the mapping device 30 and/or the evaluation device 60 into processing device 20. For example, the processing device 20 may be a computing device with a processor and a memory for storing instructions which are executed by the processor. Hence, the memory may provide instructions for causing the processor to perform the above-described operations. In this connection, processing device 20 may, communicatively coupled with the first, second and third memory 11, 12, 13, output device 40 and input device 50 via appropriate interfaces.

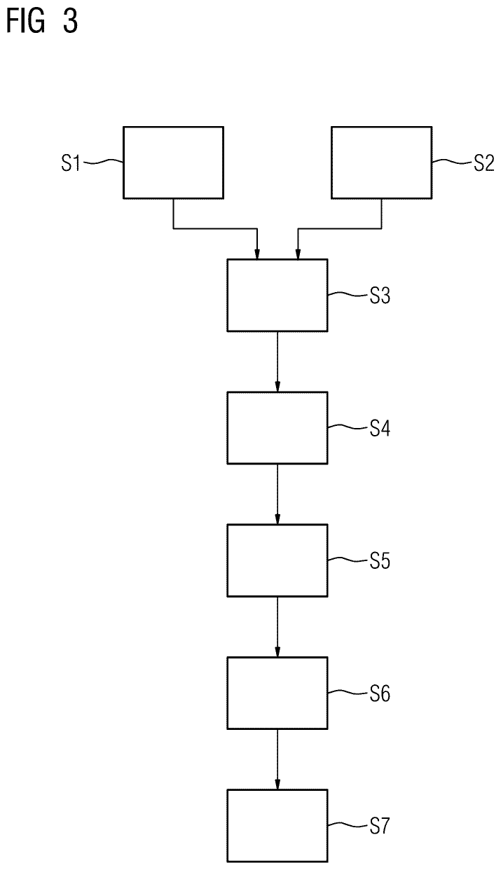

FIG. 3 shows a block diagram, illustrating a method for processing medical data according to an embodiment. The method may comprise any appropriate steps for performing operations as already described above in connection with the medical data processing apparatus 1. Furthermore, the above-described medical data processing apparatus 1 may also comprise appropriate devices or components in order to perform the method as described below.

The method of processing medical data comprises a step S1 of storing image data for a plurality of medical images. Each medical image may comprise an associated time stamp, in particular a timestamp representing the point in time when the respective medical image has been recorded.

In a step S2 at least one medical report for a corresponding stored medical image is stored. The medical report may comprise at least one documentation element for each image element in the corresponding medical image.

The method further comprises a step S3 of receiving a selection for a first medical image and/or a first medical report for the first medical image. Further, the method comprises a step S4 of receiving a selection for a second medical image.

In a step S5 a mapping between the first medical image and the second medical image is calculated. The mapping may be based on the image elements in the first and second medical image.

Further, the method comprises a step S6 of determining an image element in the second medical image for which a documentation element exists in the first medical report. For this purpose a position, size and/or shape of the image element in the second image may be determined using the mapping between the first medical image and the second medical image.

Finally, the method comprises a step S7 of generating a draft second medical report for the second medical image, wherein a documentation element for the second medical report is automatically generated using the corresponding documentation element for the corresponding image element in the first medical report.

Summarizing, embodiments of the present invention relate to a processing of medical data, in particular image data of medical images and related medical reports for an automated generation of a draft report for a newly recorded medical image. It is for this purpose that the image elements relating to corresponding documentation elements for a former medical image are mapped to image elements in the newly recorded medical image. Based on this mapping, an automated generation of the draft for the medical report of the newly recorded medical image is performed.

Wherever not already described explicitly, individual embodiments, or their individual aspects and features, de-scribed in relation to the drawings can be combined or ex-changed with one another without limiting or widening the scope of the described invention, whenever such a combination or exchange is meaningful and in the sense of this invention. Advantages which are described with respect to a particular embodiment of present invention or with respect to a particular figure are, wherever applicable, also advantages of other embodiments of the present invention.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (Soc), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

Although the present invention has been shown and described with respect to certain example embodiments, equivalents and modifications will occur to others skilled in the art upon the reading and understanding of the specification. The present invention includes all such equivalents and modifications and is limited only by the scope of the appended claims.

What is claimed is:

1. A medical data processing apparatus, comprising:
a first storage device configured to store image data for a plurality of medical images, each of the plurality of medical images having an associated time stamp;
a second storage device configured to store at least one medical report for a corresponding medical image stored in the first storage device, the at least one medical report including at least one documentation element for an image element in the corresponding medical image;
an input device configured to receive a selection of at least one of a first medical image or a first medical report for the first medical image, and configured to receive a selection of a second medical image;
a mapping device configured to calculate a mapping between the first medical image and the second medical image; and
a processing device configured to determine at least one image element in the second medical image for which a corresponding documentation element is included in the first medical report, wherein the determination is performed using the mapping between the first medical image and the second medical image,
wherein the processing device includes a documentation module configured to generate a documentation element for a draft second medical report for the second medical image, and
wherein the documentation element for the draft second medical report is automatically generated using the corresponding documentation element for the image element in the first medical report.

2. The medical data processing apparatus according to claim 1,
wherein at least one of
the medical data processing apparatus includes an output device configured to output the first medical image, the first medical report for the first medical image, the second medical image and the draft second medical report,
the input device is configured to receive user input for a selection of at least one of an image element in output medical images or a documentation element in output medical reports, or
the processing device includes a processing module configured to identify an image element corresponding to the selection in each of the first medical image and the second medical image, and to identify a documentation element corresponding to one of the first medical report or the draft second medical report; and
wherein the output device is further configured to highlight the identified image elements in the first medical image and the second medical image and the identified documentation elements in the first medical report and the draft second medical report.

3. The medical data processing apparatus according to claim 1,
wherein the input device is configured to receive an adjustment for a mapping of an image element in the first medical image and an image element in the second medical image, and
wherein the processing device is configured to adjust the mapping between the image element in the first medical image and the image element in the second medical image based on the adjustment.

4. The medical data processing apparatus according to claim 1, further comprising:
a third storage device configured to at least one of
store the mapping between the first medical image and the second medical image, or
store a mapping between a pixel in the first medical image and a corresponding pixel in the second medical image.

5. The medical data processing apparatus according to claim 1, wherein the processing device comprises:
an analysis module configured to determine at least one property of the at least one image element in the second medical image, and wherein
the documentation module is configured to generate the draft second medical report using the at least one property of the at least one image element.

6. The medical data processing apparatus according to claim 5, wherein the analysis module is configured to determine the at least one property using a semantic analysis of the corresponding documentation element in the first medical report.

7. The medical data processing apparatus according to claim 1, further comprising:
an evaluation device configured to
identify a related image element in a plurality of medical images and a respective property of each related image element, and
determine a progression of the respective property of the related image element as a function of the associated time stamps.

8. The medical data processing apparatus according to claim 1, wherein the documentation module is configured to generate a respective documentation element in the draft second medical report for at least one of (i) each image element in the first medical image or (ii) each documentation element in the first medical report.

9. The medical data processing apparatus according to claim 1, further comprising:
a first data interface configured to be coupled to a medical diagnostic imaging system to receive data of medical images, and to store the data of medical images in the first storage device.

10. The medical data processing apparatus according to claim 1, further comprising:
a second data interface configured to at least one of
provide data of a medical image and a corresponding medical report to an external processing device, or
receive information for an image element of a medical image or for a documentation element of a medical report, and automatically add the received information to the at least one of the medical image or medical report.

11. The medical data processing apparatus according to claim 1, further comprising:
a collation device configured to
identify at least one documentation element in the first medical report using a semantic analysis of the first medical report, and
determine a corresponding image element in the first medical image for each of the at least one identified documentation element.

12. The medical data processing apparatus according to claim 1, wherein at least one of
a plurality of image elements are positioned within the first medical image or the second medical image at a related embedded location, or
the plurality of image elements include an indication of at least one of a location, an organ, a lesion or a size indicator.

13. The medical data processing apparatus according to claim 1, wherein the mapping between the first medical image and the second medical image includes a mapping of two-dimensional or three-dimensional coordinates.

14. A method of processing medical data, the method comprising:
storing image data for a plurality of medical images, each of the plurality of medical images having an associated time stamp;
storing at least one medical report for a corresponding stored medical image, the at least one medical report including at least one documentation element for each image element in the corresponding stored medical image;
receiving a selection of at least one of a first medical image or a first medical report for the first medical image;
receiving a selection of a second medical image;
calculating a mapping between the first medical image and the second medical image;
determining an image element in the second medical image for which a corresponding documentation element exists in the first medical report, wherein at least one of position, size or shape of the image element in the second medical image is determined using the mapping between the first medical image and the second medical image; and
generating a draft second medical report for the second medical image, wherein a documentation element for the draft second medical report is automatically generated using the corresponding documentation element for the image element in the first medical report.

15. A non-transitory computer program product including a computer program having program code sections for causing a control device to execute the method according to claim 14 when the computer program is executed at the control device.

16. The medical data processing apparatus according to claim 2, further comprising:
a collation device configured to
identify at least one documentation element in the first medical report using a semantic analysis of the first medical report, and
determine a corresponding image element in the first medical image for each of the at least one identified documentation element.

17. The medical data processing apparatus according to claim 2,
wherein the input device is configured to receive an adjustment for a mapping of an image element in the first medical image and an image element in the second medical image, and
wherein the processing device is configured to adjust the mapping between the image element in the first medical image and the image element in the second medical image based on the adjustment.

18. The medical data processing apparatus according to claim 2, wherein the processing device comprises:
an analysis module configured to determine at least one property of the at least one image element in the second medical image, and wherein
the documentation module is configured to generate the draft second medical report using the at least one property of the at least one image element.

19. The medical data processing apparatus according to claim 2, further comprising:
an evaluation device configured to
identify a related image element in a plurality of medical images and a respective property of each related image element, and
determine a progression of the respective property of the related image element as a function of the associated time stamps.

20. The medical data processing apparatus according to claim 6, further comprising:
a collation device configured to
identify at least one documentation element in the first medical report using a semantic analysis of the first medical report, and
determine a corresponding image element in the first medical image for each of the at least one identified documentation element.
